(19) United States
(12) Patent Application Publication
(10) Pub. No.: US 2021/0322618 A1
(43) Pub. Date: Oct. 21, 2021

(54) Adjusting electronic fragrance emitter and method

(71) Applicants: Korri L. Wright, Yuma, AZ (US); Aaron B. Moncur, Mesa, AZ (US); Michael L. Hadley, Mesa, AZ (US); Brandon M. Graham, Chandler, AZ (US); John B. Martell, Gilbert, AZ (US)

(72) Inventors: Korri L. Wright, Yuma, AZ (US); Aaron B. Moncur, Mesa, AZ (US); Michael L. Hadley, Mesa, AZ (US); Brandon M. Graham, Chandler, AZ (US); John B. Martell, Gilbert, AZ (US)

(21) Appl. No.: 16/853,553

(22) Filed: Apr. 20, 2020

(51) Int. Cl. A61L 9/03; B05B 9/00; B05B 9/03; B05B 9/01

(52) U.S. Cl. CPC A61L 9/037; B05B 9/002; A61L 2209/15; B05B 9/01; A61L 2209/133; B05B 9/03

(57) ABSTRACT

The device includes a substantially L-shaped body including a docking station for mounting a wick-based fragrance dispensing bottle within a range of sizes. The docking station can include a number of resilient support tabs that grasp the neck of the bottle and serve to localize the bottle's top wick to a central plane regardless of bottle size so that it can be adjustably engaged by a movable heater. Resilient guide tabs straddling the plane are stiffer than a resilient latching tab that moves within the plane. The heater can be mounted on a sliding carriage that can move along the plane to closely nest the wick for increased vapor emission and move away to decrease vapor emission.

### FIELD OF THE INVENTION

This invention relates to vaporizers, and more particularly to electrically activated vaporizing fragrance emitters.

### BACKGROUND

Various devices have long been used in households to provide pleasant smells. More recently, electronic vaporizers such as the type shown in Ruffolo et al., U.S. Pat. No. 6,236,807 have been developed to dispense a more uniformly intense fragrance by vaporizing a fragrance infused liquid from a container such as a bottle having a sealed upper spout through which passes a wick which, through capillarity, draws up an amount of liquid from the bottle. The upper tip of the wick is exposed to a heater which volatilizes the liquid into the ambient air.

The bottle carrying the fragrant liquid can be mounted to the emitter through a number of means including a screw-type receptacle which engages threads on the outer surface of the spout of the bottle. Unfortunately, such a screw-type threaded engagement can limit the range of bottles which will properly engage the threads.

In order to address the variability of bottle sizes, Yip et al., U.S. Pat. No. 6,950,607 provides a spring-loaded clamp system for grasping the neck of a bottle having a diameter of between about 14 mm and 24 mm.

Yip et al. uses movable blocking plates to adjust the amount of vapor exiting the device. This can be inefficient because the liquid in the bottle is still exhausted within roughly the same amount of time, even though not all vapor has exited the housing. It is assumed some vapor may condense in the inner parts of the device when ventilation is restricted by the blocking plates.

Therefore, there is a need for an electronic fragrance emitters which addresses one or more of the above problems or inadequacies.

### SUMMARY

The principal and secondary objects of the invention are to provide an improved electronic fragrance emitter. These and other objects are achieved by providing an emitter including an automatically adjustable container mounting structure.

In some embodiments there is provided an apparatus for emitting a fragrance, said apparatus comprises: a housing; a container carrying a supply of liquid fragrance, wherein said container comprises a wick having a substantially vertical axis; a heater movably mounted to said housing; wherein said heater comprises an electrical heating element; a docking station located on said housing releasably mounting said container so that said wick is intersected by a vertical plane intersecting said housing; wherein said docking station comprises a first support tab movable along said plane.

In some embodiments said housing comprises a vent intersected by said plane.

In some embodiments said first support tab is biased toward a first radially inward position on said plane.

In some embodiments said first support tab comprises: a first cantilevered beam having a fixed end secured to said housing, and a free end; a hook structure extending radially inwardly with respect to said axis from said free end; wherein said hook structure comprises a concave arcuate tip oriented to bear against said container.

In some embodiments said docking station further comprises: a pair of angularly spaced apart guide tabs substantially straddling said plane; wherein each of said guide tabs is biased toward said axis.

In some embodiments said guide tabs are substantially equidistantly angularly spaced apart from said plane.

In some embodiments said guide tabs are angularly spaced apart from one another at an angle of between 110 degrees and 130 degrees.

In some embodiments said guide tabs have a substantially equivalent resiliency whereby their combined biasing force acting upon said bottle drives said wick toward a position on said plane.

In some embodiments said first support tabs and said guide tabs mount said bottle in absence of any additional structure contacting said bottle.

In some embodiments said first cantilevered beam can deflect angularly at least 5 degrees.

In some embodiments said apparatus further comprises: a second support tab; a third support tab; wherein each of said support tabs is resiliently cantileveredly connected to said docking station; and, wherein said first, second and third support tabs are angularly spaced apart from one another.

In some embodiments said first, second and third movable support tabs are symmetric about a vertical plane bisecting said body and said container.

In some embodiments said heater further comprises a carriage structure slidingly mounted to said housing and moveable in a direction along said plane.

In some embodiments said carriage structure can move between a first position proximal to said wick and a second position distal from said wick.

In some embodiments said first position and said second position are located on said plane.

In some embodiments said apparatus further comprises a hand-manipulable button connected to said carriage and extending from a surface of said body for moving said carriage between said first and second positions.

In some embodiments said heater further comprises: a ceramic cradle in thermal communication with said heating element; wherein said cradle comprises a substantially U-shaped open-ended nook formed between a pair of spaced apart arms; wherein said open end is oriented along said plane; wherein said arms straddle said axis while said heater is located in a first position and do not straddle said axis while said heater is located in a second position.

In some embodiments there is provided that in an electronic fragrance emitter for mounting an heating the wick of a fragrant liquid-carrying container, an improvement which comprises: a bottle mounting station; at least one radially movable support tab oriented to engage a groove on said container; a carriage comprising a heater element; said carriage being movable between a first location disengaging said wick and a second location engaging said wick;

In some embodiments there is provided a method for mounting a fragrance container to an electronic fragrance emitter, said method comprises: removing a first liquid fragrance container having a first neck diameter from a docking station on said emitter; selecting a second liquid fragrance container having a second neck diameter different from said first neck diameter; and, inserting said second liquid fragrance container into said docking station; wherein said inserting comprises: urging said second liquid fragrance container toward a plane intersecting said emitter; wherein said urging comprises: guiding said second liquid fragrance container using a pair of resilient guide tabs straddling said plane; latching said second liquid fragrance container using a support tab biased in a direction along said plane and opposing said guide tabs.

The text of the claims is incorporated herein by reference as describing features in some embodiments.

### BRIEF DESCRIPTION OF THE DRAWINGS

FIG. 1 is a diagrammatic perspective view of an electronic fragrance emitter and mounted bottle according to an exemplary embodiment of the invention.

FIG. 2 is a diagrammatic perspective view of the electronic fragrance emitter showing a vertical bisecting plane.

FIG. 3 is a diagrammatic cross-sectional side view of liquid fragrance container.

FIG. 4 is a diagrammatic cross-sectional side view the electronic fragrance emitter of FIG. 3 detailing the heater.

FIG. 5 is a diagrammatic perspective exploded view of the electronic fragrance emitter of FIG. 3.

FIG. 6 is a diagrammatic cross-sectional side view the electronic fragrance emitter of FIG. 3 detailing the docking station.

FIG. 7 is a diagrammatic bottom view of the electronic fragrance emitter of FIG. 3.

FIG. 8 is a diagrammatic bottom view of the support tabs.

FIG. 9 is a diagrammatic cross-sectional side view the electronic fragrance emitter of

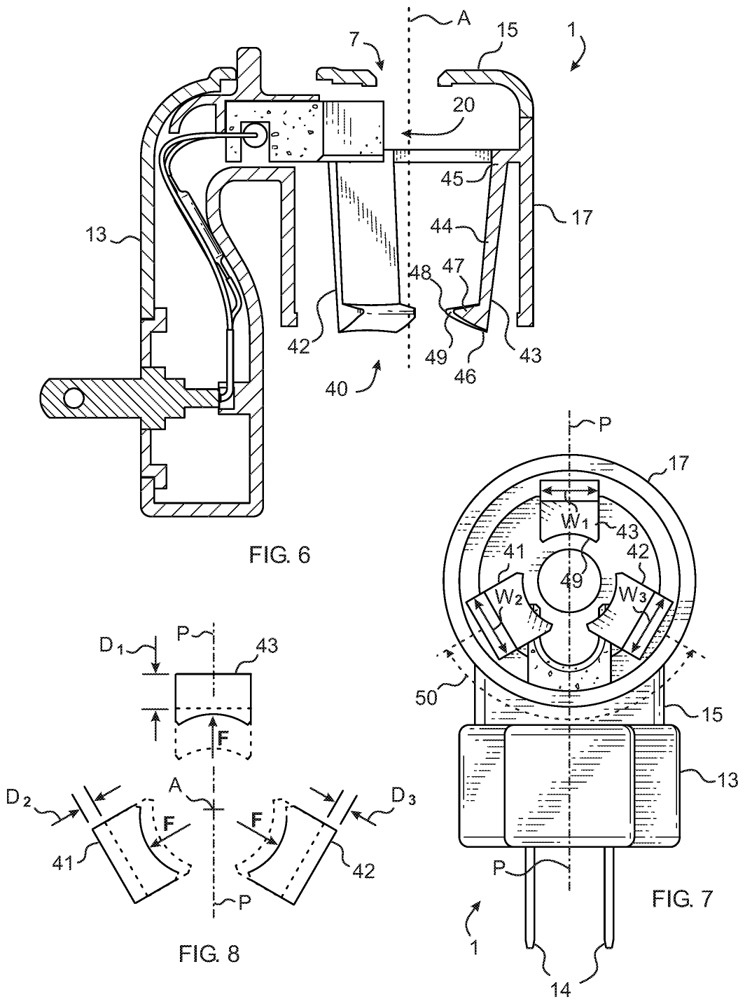

FIG. 3 mounting a small bottle.

FIG. 10 is a diagrammatic cross-sectional side view the electronic fragrance emitter of FIG. 3 mounting a large bottle.

FIG. 11 is a flow chart diagram of method for mounting a fragrance container to an electronic fragrance emitter according to an exemplary embodiment of the invention.

FIG. 12 shows the top, front, right side perspective view of an ornamental design of an electronic liquid fragrance emitter according to an exemplary embodiment of the invention.

FIG. 13 shows the bottom, back, left side perspective view thereof.

FIG. 14 shows the front elevation view thereof.

FIG. 15 shows the back elevation view thereof.

FIG. 16 shows the top plan view thereof.

FIG. 17 shows the bottom plan view thereof

FIG. 18 shows the right side elevation view thereof, the left side elevation view being a mirror image thereof

FIG. 19 shows the top, front, right side perspective view of an ornamental design of an electronic liquid fragrance emitter according to an alternate exemplary embodiment of the invention.

FIG. 20 shows the bottom, back, left side perspective view thereof.

FIG. 21 shows the front elevation view thereof.

FIG. 22 shows the back elevation view thereof.

FIG. 23 shows the top plan view thereof.

FIG. 24 shows the bottom plan view thereof.

FIG. 25 shows the right side elevation view thereof, the left side elevation view being a mirror image thereof.

### DESCRIPTION OF THE EXEMPLARY EMBODIMENTS

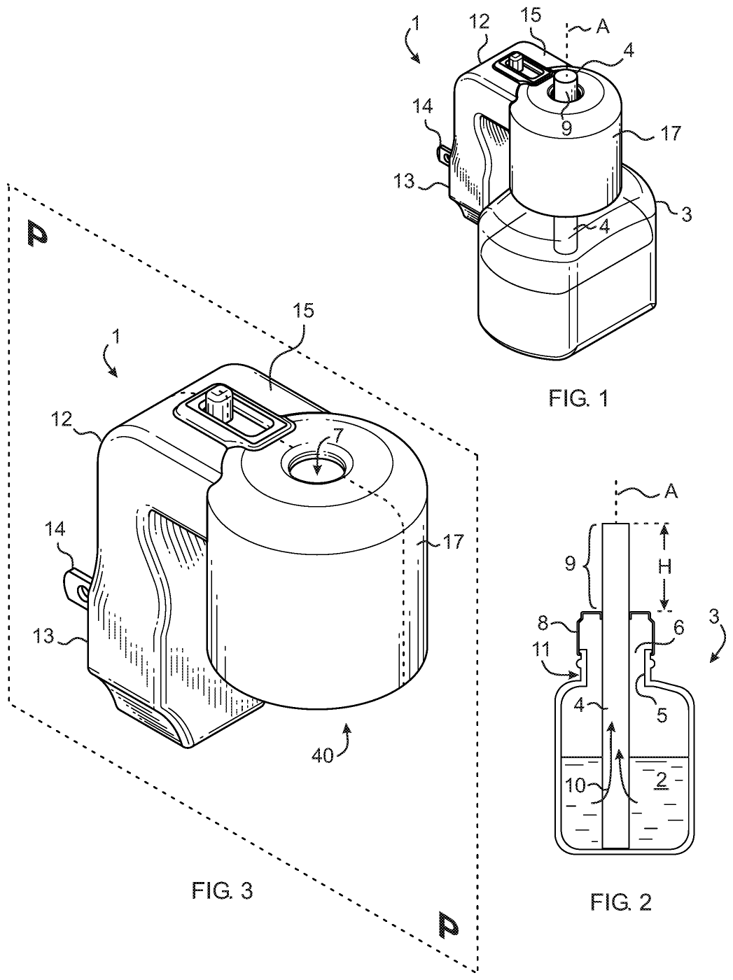

In this specification, the references to top, bottom, upward, downward, upper, lower, above, below, vertical, horizontal, sideways, lateral, back, front, etc. can be used to provide a clear frame of reference for the various structures with respect to other structures while the fragrance emitter is oriented to be installed on a wall-mounted electrical socket as shown in FIG. 1, and not treated as absolutes when the frame of reference is changed, such as when the emitter is resting on the ground unused.

The term “substantially” can be used in this specification because manufacturing imprecision and inaccuracies can lead to non-symmetricity and other inexactitudes in the shape, dimensioning and orientation of various structures. Further, use of “substantially” in connection with certain geometrical shapes, letter shapes, such as “U-shaped” and orientations, such as “parallel” and “perpendicular”, can be given as a guide to generally describe the function of various structures, and to allow for slight departures from exact mathematical geometrical shapes, letter shapes, and orientations, while providing adequately similar function. Those skilled in the art will readily appreciate the degree to which a departure can be made from the mathematically exact geometrical references.

If used in this specification, the word “axial” is meant to refer to directions, movement, or forces acting substantially parallel with or along a respective axis, and not to refer to rotational nor radial nor angular directions, movement or forces, nor torsional forces unless otherwise noted.

In this specification the units “millimeter” or “millimeters” can be abbreviated “mm”.

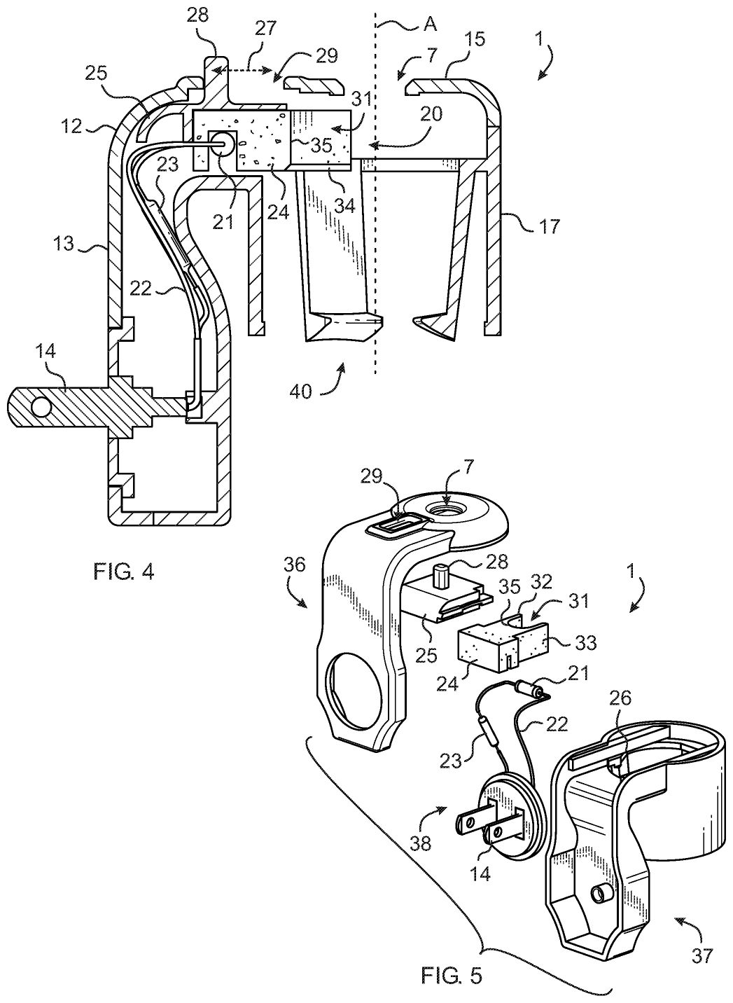

Referring now to FIGS. 1-3 there is shown an electronic fragrance emitter 1 for volatilizing an amount of flagrance-carrying liquid 2 such as scented oil from a container such as a bottle 3 having a relatively narrow cylindrical upper neck 5 surrounding an upper opening 6 which is sealed by a cap 8. A circumferential groove 11 can be formed at the base of the neck between the cap and the bottom chamber of the bottle. A wick 4 contacts the liquid inside the bottle and extends upwardly along a substantially vertical axis A through an opening in the cap. A substantially cylindrical upper end region 9 of the wick can extend vertically a distance H above the cap. The wick is made from a liquid permeable material which through capillarity draws 10 an amount of the scented liquid up out of the lower chamber of the bottle into the upper end region. A heater in the emitter heats the upper end region of the wick which volatilizes some of the liquid to form a fragrant vapor which passes out of the emitter through an upper hole 7 in proximity to the upper end region.

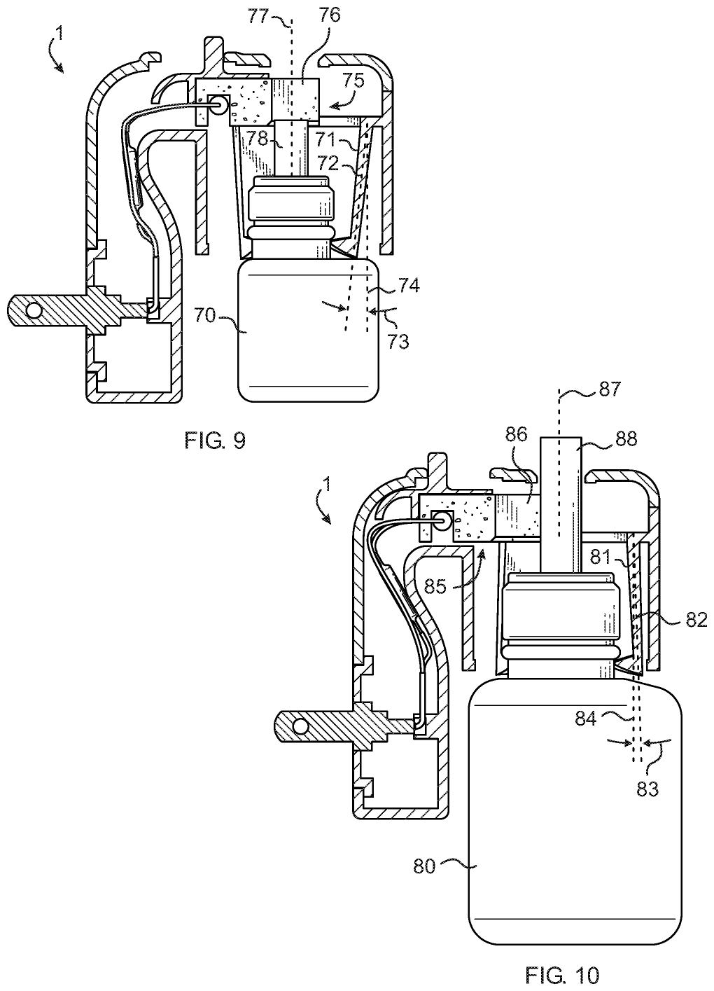

Referring primarily to FIGS. 3-4, the emitter 1 can include a substantially inverted L-shaped housing 12 made from a solid, durable material such as hard plastic. A substantially vertical back portion 13 can include electrical contacts 14 for engaging the standard household wall outlet and supplying power to the heater 20 movably mounted within the housing. A top portion 15 of the housing can extend substantially orthogonally and forwardly from the top end of the back portion along a substantially vertical plane P intersecting the housing. In this embodiment the plane substantially bisects the housing and is in alignment with the axis A of the wick. The top portion can include a substantially cylindrical hood 17 carrying a docking station 40 for releasably mounting the bottle 3 to the emitter. The hood can include a substantially circular upper hole 7 substantially aligned with the vertical axis of the wick to act as a vent for allowing volatilized scented liquid to escape from the housing. The hole can be located on the plane so that it is oriented near the wick. Indeed, for some bottles the wick can extend through the hole as shown in FIG. 1.

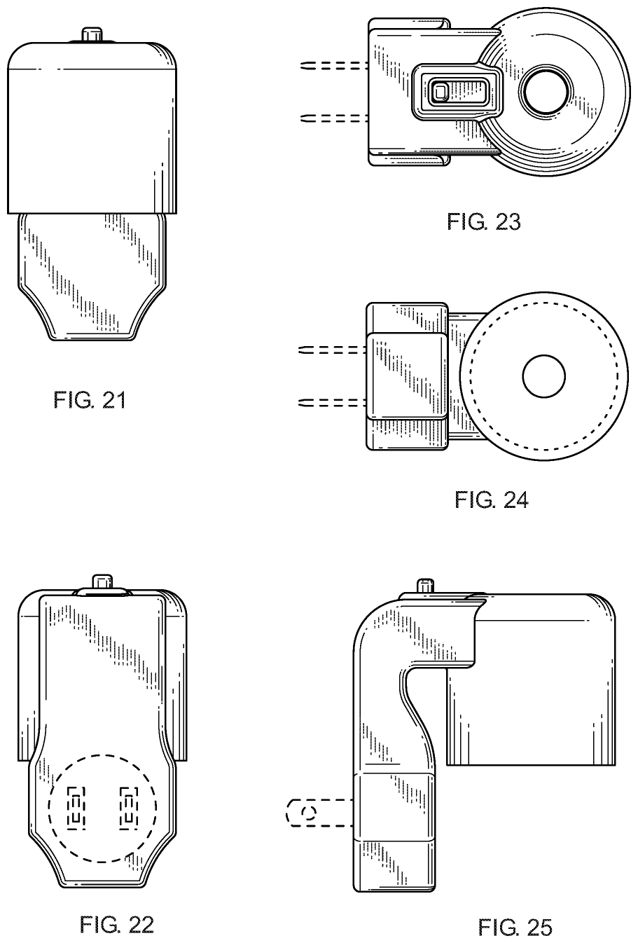

As shown in FIGS. 3-5, the heater 20 can be mounted within the housing 12 at a location which is proximate to the location of the wick of a mounted fragrance bottle. The heater can include an electrical heating element 21 wired 22 to the electrical contacts 14. A fuse 23 can prevent overheating of the element. The element can be mounted within a ceramic cradle 24 in thermal communication with the heating element. The cradle can be secured to a carriage 25 which itself can be slidingly mounted to a track 26 in the housing. The carriage can slide back and forth in directions 27 aligned with the plane P between a forward position (depicted in FIG. 9) and a rearward position (depicted in FIG. 4). In this way the carriage can move radially with respect to the axis A of the wick. The positioning of the carriage adjusts the proximity of the heated cradle to the wick, and thereby adjusts the temperature of the wick, and consequently the rate at which the scented liquid is volatilized. The sliding motion and position of the carriage can be manually accomplished by a user sliding a button 28 attached to the carriage and extending upwardly through a slot 29 in the housing. The carriage can be positioned discretely at either the forward or rearward positions, or at variable positions in between.

The ceramic cradle 24 can include a substantially U-shaped, open-ended nook 31 formed between a pair of spaced apart arms 32,33. The nook can be oriented so that its open end opens in the direction of the plane P and toward the position of the wick. Further, the nook can be sized to nest the wick when the carriage 25 is in its forward position so that the arms straddle the wick and provide heat to a greater surface area of the wick increasing volatilization. The laterally deepest part of the nook can have a substantially cylindrically shaped sidewall 35 to more closely nest the substantially cylindrical upper end portion of the wick. The length of the arms and thus the depth of the nook can be selected so that the arms do not straddle the wick while the carriage is located in its rearward position. The bottom edge of the nook can include a beveled surface 34 to facilitate vertical insertion of the wick into the nook as may occur when a bottle is mounted while the carriage is in or near its forward position.

In this way the lateral position of the heater can be adjusted along the plane to more closely engage the wick in order to adjust the amount of heat being imparted on the wick and thereby adjust the intensity of the emitted fragrance. In addition, this function allows for flexibility in the positioning of the wick along the plane for those bottle necks having slightly different dimensioning.

Although the above exemplary embodiment uses a hand-manipulable heater carriage, it shall be understood that the emitter can be adapted to include automated and/or motorized elements.

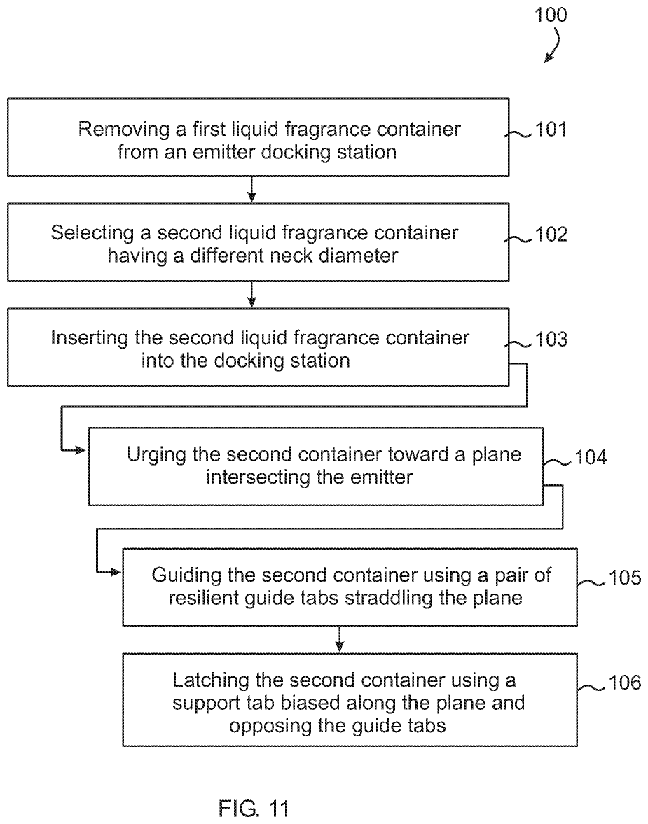

FIG. 5 also shows that the housing can be formed by two matable injection molded shells 36,37 that do not require further machining A separately assembled electrical harness 38 can include all the necessary electronic components.

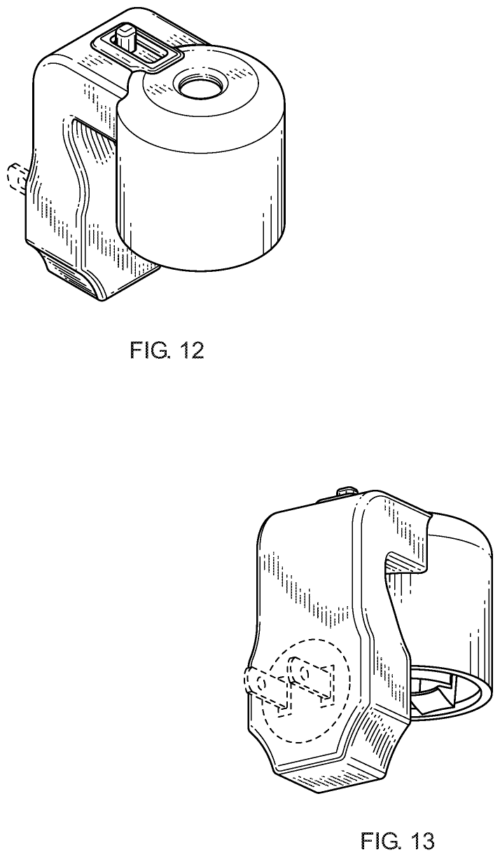

Referring primarily to FIGS. 6-8, the emitter can include a docking station 40 shaped and dimensioned to grasp the bottle and hold it in place during operation. The docking station can include one or more resilient support tabs 41,42,43 that engage the bottle and bear against the bottle surfaces to hold it in place until the bottle is removed. The three support tabs temporarily engage the circumferential groove at the base of the neck of the bottle. Each support tab can include an oblong beam 44 cantileveredly connected at a first fixed end 45 to the emitter housing. An opposite free end 46 can have a radially inwardly projecting hooking prominence simply referred to as a hook 47 that extends substantially orthogonally to the beam. The hook can be shaped, dimensioned and oriented to snugly engage the groove at the base of the neck of the bottle. As viewed from the side, the hook can have a rounded tip 48 to allow it to more easily slip over structures surrounding the bottle groove during mounting and de-mounting of the bottle. As viewed from below the tip can be formed into a concave arcuate face 49 for intimately engaging the correspondingly convex circular groove over an angular distance.

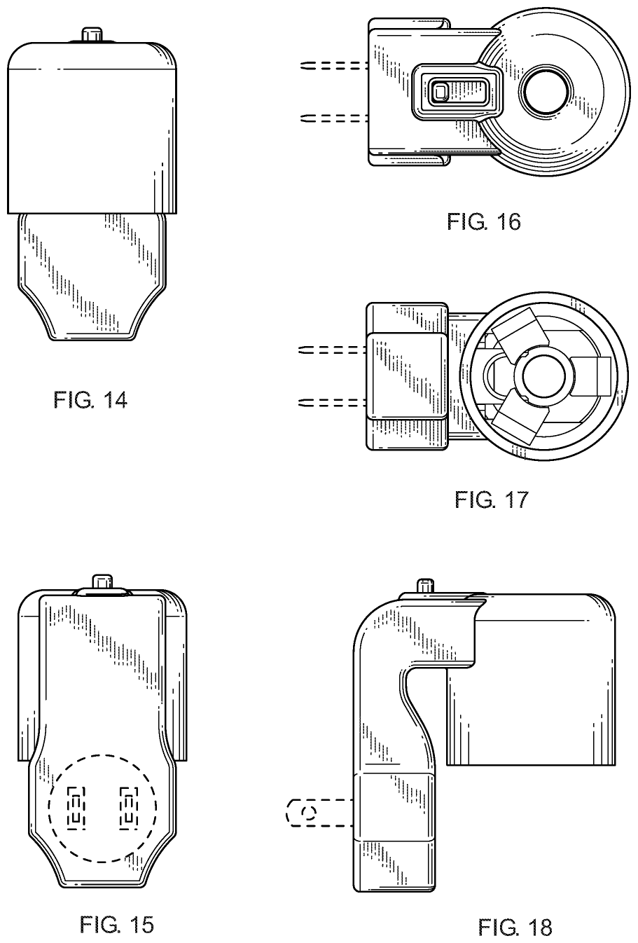

As shown in FIG. 7, the three support tabs 41,42,43 can be divided into two groups. The first group can consist of the first two tabs which can act as guide tabs 41,42. The second group can consist of the third tab which can act as a latching tab 43. The two guide tabs can substantially straddle the substantially central plane P of the emitter while locking tab is intersected by the plane. Further, the guide tabs can be substantially equidistantly angularly spaced apart from said plane. The guide tabs can be angularly spaced apart from one another at an angle 50 of between 90 degrees and 150 degrees, more preferably between 110 degrees and 130 degrees, and most preferably about 120 degrees. Greater degrees of angular separation can leave an angular gap between the guide tabs which may not adequately engage smaller bottles, whereas a smaller angular gap may not provide superior centering over a range of bottle sizes. The guide tabs 41,42 can be more rigid than the latch tab 43 in order to urge the bottle toward the central plane P. For example, each guide tab can have a larger width dimension W2,W3 which is larger than the width dimension W1 of the latch tab. This allows the docking station and the three support tabs to be made from a single molding of injection molded material.

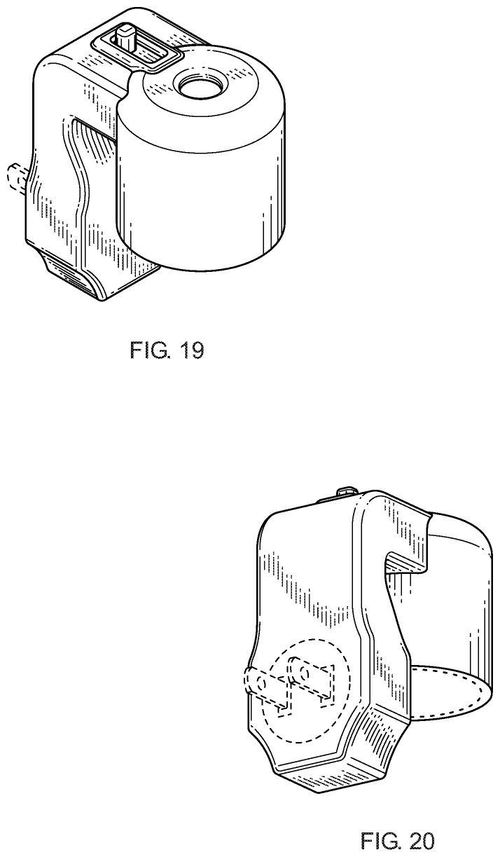

As seen from below in FIG. 8, by being more rigid, the guide tabs 41,42 deflect a lesser distance D2,D3 than the deflected distance D1 of the latch tab 43 when subjected to the same radially outward deflecting force of magnitude F. The original radially inward positions of the tabs are denoted in dashed lines whereas their resultant radially outward positions, while being subjected to the force, are denoted by solid lines.

In this way the guide tabs 41,42 forcefully urge the bottle neck and consequently the substantially vertical axis A of the wick toward being in alignment with the plane P, while the latch tab 43, being more resilient, can engage the neck at a position which is more radially outward than would be achieved with all the tabs having the same rigidity. The difference in rigidity helps overcome small manufacturing imperfections in the shape, location and dimensioning of the tabs and their resultant inaccuracies in biasing which could otherwise lead to possible improper positioning of the wick with respect to the heater.

In other words, the above features of the docking station causes the axially central wick of the mounted bottle to remain substantially radially fixed within emitter regardless of the size of the bottle being engaged. Thus, the location of the upper end portion of the wick can be predictably located for a range of bottle sizes. Indeed, the combined biasing force of the support tabs can drive the wick toward a position on the plane in absence of any additional guide structure contacting the bottle. Further, the support tabs can mount the bottle in absence of any additional structure contacting the bottle. In this way a single docking station can successively mount a plurality of fragrance bottles having differently dimensioned bottle necks in absence additional docking stations.

The above-described mechanism provides a docking station allows for simplified, snap-fit mounting and de-mounting of the bottle by simply forcefully pushing the bottle into the docking station or forcefully pulling the bottle from the station in absence of any other attachment steps or mechanisms. This simplified approach allows unskilled and untrained persons to intuitively operate the emitter.

In an alternate embodiment, the three support tabs can be substantially identical to one another in size, shape and material properties, and be evenly angularly spaced apart so that they have similar lengths, angular coverage and hook sizes to accommodate differently sized bottles while maintaining the central axis of the wick in substantial allignment with the emitter central vertical plane.

FIGS. 9-10 show that both a smaller bottle 70, and a larger bottle 80 can be separately mounted to the same emitter 1. In the case of the smaller bottle 70 the latch tab 71 is deflected only minimally, if at all from its radially inward unloaded position where the long dimension 72 of the beam forms an angle 73 with vertical 74 of about 6 degrees. In the case of the larger bottle 80, the latch tab 81 is deflected nearly to its full extent at its maximum radially outward position where the long dimension 82 of the beam forms an angle 83 with vertical 84 of about −2 degrees. Thus for this embodiment the maximum deflection of the latch tab is about 8 degrees. For most embodiments it is anticipated that the latch tab be capable of at least 5 degrees of angular deflection, more preferably at least 8 degrees, and most preferably at least 10 degrees.

FIGS. 9-10 further show that the heater can move between a first position 75 proximal to the wick, and a second position 85 more distal from the wick. In FIG. 9 the heater support arms 76 straddle the substantially central vertical axis 77 of the wick 78. In FIG. 10 the heater support arms 86 do not straddle the substantially central vertical axis 87 of the wick 88.

FIG. 11 shows the primary steps of a method 100 for mounting a fragrance container to an electronic fragrance emitter. The method includes first removing 101 a first liquid fragrance container having a first neck diameter from a docking station on the emitter. For example this could mean removing the smaller bottle 70 from the emitter 1 shown in FIG. 9. A second liquid fragrance container is selected 102 having a second neck diameter different from the neck diameter of the first bottle. For example, this could be the larger bottle 80 shown in FIG. 10. The second container can then be inserted 103 into the docking station. The insertion of the second container can include urging 104 the container toward a plane intersecting the emitter. The urging can include guiding 105 the second container using a pair of resilient guide tabs straddling the plane, and latching 106 the second container using a support tab biased in a direction along the plane and opposing the combined force of the guide tabs.

FIGS. 12-18 show the appearance of an electronic liquid fragrance emitter in support of a claim for an original ornamental design.

FIGS. 19-25 show the appearance of an alternate electronic liquid fragrance emitter in support of a claim for an original ornamental design.

While the preferred embodiments of the invention have been described, modifications can be made and other embodiments may be devised without departing from the spirit of the invention and the scope of the appended claims.

What is claimed is:

1. An apparatus for emitting a fragrance, said apparatus comprises:
a housing;
a container carrying a supply of liquid fragrance, wherein said container comprises a wick having a substantially vertical axis;
a heater movably mounted to said housing;
wherein said heater comprises an electrical heating element;
a docking station located on said housing releasably mounting said container so that said wick is intersected by a vertical plane intersecting said housing;
wherein said docking station comprises a first support tab movable along said plane.

2. The apparatus of claim 1, wherein said housing comprises a vent intersected by said plane.

3. The apparatus of claim 1, wherein said first support tab is biased toward a first radially inward position on said plane.

4. The apparatus of claim 3, wherein said first support tab comprises:
a first cantilevered beam having a fixed end secured to said housing, and a free end;
a hook structure extending radially inwardly with respect to said axis from said free end;
wherein said hook structure comprises a concave arcuate tip oriented to bear against said container.

5. The apparatus of claim 4, wherein said docking station further comprises:
a pair of angularly spaced apart guide tabs substantially straddling said plane;
wherein each of said guide tabs is biased toward said axis.

6. The apparatus of claim 5, wherein said guide tabs are substantially equidistantly angularly spaced apart from said plane.

7. The apparatus of claim 6, wherein said guide tabs are angularly spaced apart from one another at an angle of between 110 degrees and 130 degrees.

8. The apparatus of claim 7, wherein said guide tabs have a substantially equivalent resiliency whereby their combined biasing force acting upon said bottle drives said wick toward a position on said plane.

9. The apparatus of claim 5, wherein said first support tabs and said guide tabs mount said bottle in absence of any additional structure contacting said bottle.

10. The apparatus of claim 4, wherein said first cantilevered beam can deflect angularly at least 5 degrees.

11. The apparatus of claim 1, which further comprises:
a second support tab;
a third support tab;
wherein each of said support tabs is resiliently cantileveredly connected to said docking station; and,
wherein said first, second and third support tabs are angularly spaced apart from one another.

12. The apparatus of claim 11, wherein said first, second and third movable support tabs are symmetric about a vertical plane bisecting said body and said container.

13. The apparatus of claim 1, wherein said heater further comprises a carriage structure slidingly mounted to said housing and moveable in a direction along said plane.

14. The apparatus of claim 13, wherein said carriage structure can move between a first position proximal to said wick and a second position distal from said wick.

15. The apparatus of claim 14, wherein said first position and said second position are located on said plane.

16. The apparatus of claim 15, wherein said apparatus further comprises a hand-manipulable button connected to said carriage and extending from a surface of said body for moving said carriage between said first and second positions.

17. The apparatus of claim 1, wherein said heater further comprises:
a ceramic cradle in thermal communication with said heating element;
wherein said cradle comprises a substantially U-shaped open-ended nook formed between a pair of spaced apart arms;
wherein said open end is oriented along said plane;
wherein said arms straddle said axis while said heater is located in a first position and do not straddle said axis while said heater is located in a second position.

18. In an electronic fragrance emitter for mounting an heating the wick of a fragrant liquid-carrying container, an improvement which comprises:
a bottle mounting station;
at least one radially movable support tab oriented to engage a groove on said container;
a carriage comprising a heater element;
said carriage being movable between a first location disengaging said wick and a second location engaging said wick;

19. A method for mounting a fragrance container to an electronic fragrance emitter, said method comprises:
removing a first liquid fragrance container having a first neck diameter from a docking station on said emitter;
selecting a second liquid fragrance container having a second neck diameter different from said first neck diameter; and,
inserting said second liquid fragrance container into said docking station;
wherein said inserting comprises:
urging said second liquid fragrance container toward a plane intersecting said emitter;
wherein said urging comprises:
guiding said second liquid fragrance container using a pair of resilient guide tabs straddling said plane;
latching said second liquid fragrance container using a support tab biased in a direction along said plane and opposing said guide tabs.
